(19) United States
(12) Patent Application Publication
(10) Pub. No.: US 2025/0169479 A1
(43) Pub. Date: May 29, 2025

(54) BILIARY ATRESIA MODEL

(71) Applicant: Saint Louis University, St. Louis, MO (US)

(72) Inventors: Ajay Kumar Jain, St. Louis, MO (US); Kento Kurashima, St. Louis, MO (US)

(21) Appl. No.: 18/962,885

(22) Filed: Nov. 27, 2024

Related U.S. Application Data
(60) Provisional application No. 63/603,995, filed on Nov. 29, 2023.

(51) Int. Cl. A01K 67/027

(52) U.S. Cl. CPC A01K 67/027; A01K 2207/20; A01K 2227/108; A01K 2267/03

(57) ABSTRACT

The present disclosure relates to animal models simulating human biliary atresia. The model includes a large animal treated with an agent causing sclerosis of intrahepatic bile ducts in the animal to simulate human bile duct injury.

### CROSS REFERENCE TO RELATED APPLICATIONS

This application claims priority to and benefit of U.S. Provisional Patent Application Ser. No. 63/603,995, filed on Nov. 29, 2023, the disclosure of which is hereby incorporated by reference in its entirety.

### BACKGROUND OF THE DISCLOSURE

The present disclosure generally relates to methods and compositions for simulating human biliary atresia. The methods include simulating biliary atresia in an animal model. The compositions include an animal model simulating biliary atresia, and more particularly, a large animal model for simulating humans biliary atresia.

Biliary atresia (BA) is a rare and serious congenital disorder affecting the bile ducts in infants. In normal digestive systems, bile ducts help transport bile from the liver to the gallbladder and small intestine, aiding in the digestion of fats. However, in biliary atresia, the ducts become inflamed, damage, or blocked, leading to impaired bile flow. If left untreated, biliary atresia can lead to liver damage, cirrhosis, and eventually liver failure.

The primary treatment for biliary atresia is a surgical procedure called the Kasai procedure, where the damaged bile ducts are replaced with a section of the infant's own small intestine. Despite this treatment, many infants may eventually require a liver transplant due to ongoing liver damage.

Research into biliary atresia is crucial for understanding its underlying mechanisms, developing effective treatments, and improving patient outcomes. Current biliary atresia models use bile duct ligation which does not recapitulate human biliary atresia. The hepatic fibrosis in these models is also unlike human disease. Additionally, current small animal models cannot provide enough room for the Kasai procedure to be performed. Thus, these existing biliary atresia models lack accuracy and reproducibility in mimicking the disease progression of biliary atresia in humans.

Accordingly, there exists a need for animal models simulating human liver disease.

### BRIEF SUMMARY

In one aspect, the present disclosure is directed to a large animal model simulating human biliary atresia comprising: a large animal having sclerosis of intrahepatic bile ducts caused by administration of an agent causing bile duct injury.

In one aspect, the present disclosure is directed to an animal model simulating human biliary atresia comprising an animal having sclerosis of intrahepatic bile ducts following injection of an agent into the intrahepatic bile ducts of the animal.

In one aspect, the present disclosure is directed to a method of generating an animal model simulating human bile duct injury, the method comprising: providing an animal; and administering an agent to the animal, wherein the agent causes sclerosis of intrahepatic bile ducts in the animal thereby simulating human bile duct injury.

In one aspect, the present disclosure is directed to an animal model produced by the method of generating an animal model simulating human bile duct injury, the method comprising: providing an animal; and administering an agent to the animal, wherein the agent causes sclerosis of intrahepatic bile ducts in the animal thereby simulating human bile duct injury.

In one aspect, the present disclosure is directed to a method of generating an animal model simulating human biliary atresia, the method comprising: providing an animal; and injecting an agent into intrahepatic bile ducts of the, wherein the agent causes sclerosis of the intrahepatic bile ducts thereby simulating human biliary atresia.

In one aspect, the present disclosure is directed to an animal model produced by the method of generating an animal model simulating human biliary atresia, the method comprising: providing an animal; and injecting an agent into intrahepatic bile ducts of the, wherein the agent causes sclerosis of the intrahepatic bile ducts thereby simulating human biliary atresia.

### BRIEF DESCRIPTION OF THE DRAWING

The patent or application file contains at least one drawing executed in color. Copies of this patent or patent application publication with color drawing(s) will be provided by the Office upon request and payment of the necessary fee.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

FIG. 1 depicts photographs of the Kasai surgical procedure performed 6-week after the biliary atresia through targeted endothelial destruction (“BATTED”) procedure.

FIG. 2 are graphs showing serum chemistry of total bilirubin, conjugated-bilirubin, and GGT in the three groups of piglets that underwent sham, BATTED, or BATTED plus Kasai procedure. Bilirubin and GGT levels increased after BATTED procedure, then decreased after Kasai procedure. In one Kasai piglet in particular, the normal bilirubin level was restored after Kasai procedure. These changes are compatible to human biliary atresia.

FIG. 3 are graphs showing serum chemistry of bile acid, ALT, and ALP in the three groups of piglets that underwent sham, BATTED, or BATTED plus Kasai procedure. Bile acid level increased after BATTED procedure and then decreased after Kasai procedure. These changes are compatible to human biliary atresia.

FIG. 4 are graphs showing gene expression level related to bile acid metabolism. Examination of these genes was analyzed to elucidate the change of bile acid metabolism caused in cholestatic liver disease like biliary atresia. CYP7A1 codes the enzyme controlling the rate limiting step of bile acid synthesis and was upregulated in BATTED and plus Kasai (+Kasai) piglets. FXR is a receptor of bile acid and control bile acid synthesis and excretion. FXR was upregulated in BATTED piglet and one plus Kasai piglet showed downregulation after Kasai procedure. BSEP codes a transporter to excrete bile acid into bile canaliculus. SHP receives a signal from FXR and controls CYP7A1. CAR is controlled by FXR and controls bile acid excretion to blood. BSEP, SHP and CAR were upregulated in BATTED piglet and suppressed in plus Kasai piglet.

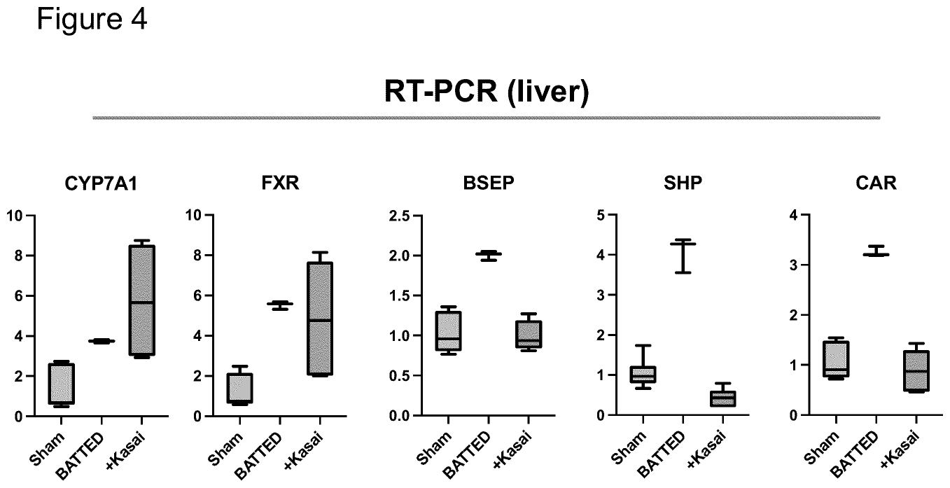

FIG. 5 shows histological change in BATTED and Kasai piglets. CK7 is a marker for bile duct and BATTED piglets exhibited bile duct proliferation as observed in human biliary atresia. Kasai piglet showed mild bile duct proliferation.

FIG. 6 shows longitudinal histological changes and CK7 expression in piglets. BATTED piglets showed severe liver fibrosis and inflammatory cell infiltration as observed in human biliary atresia. Following Kasai, piglets showed mitigation of the histological change and reduction of CK7 expression.

FIG. 7 shows longitudinal histological changes and Sirius red staining of liver collagen in piglets. BATTED piglets showed severe liver fibrosis and inflammatory cell infiltration as observed in human biliary atresia. Following Kasai, piglets showed mitigation of the histological change and reduction of Sirius red staining.

FIG. 8 shows longitudinal histological changes and COL1A1 expression in piglets. BATTED piglets showed severe liver fibrosis and inflammatory cell infiltration as observed in human biliary atresia. Following Kasai, piglets showed mitigation of the histological change and reduction of COL1A1 expression.

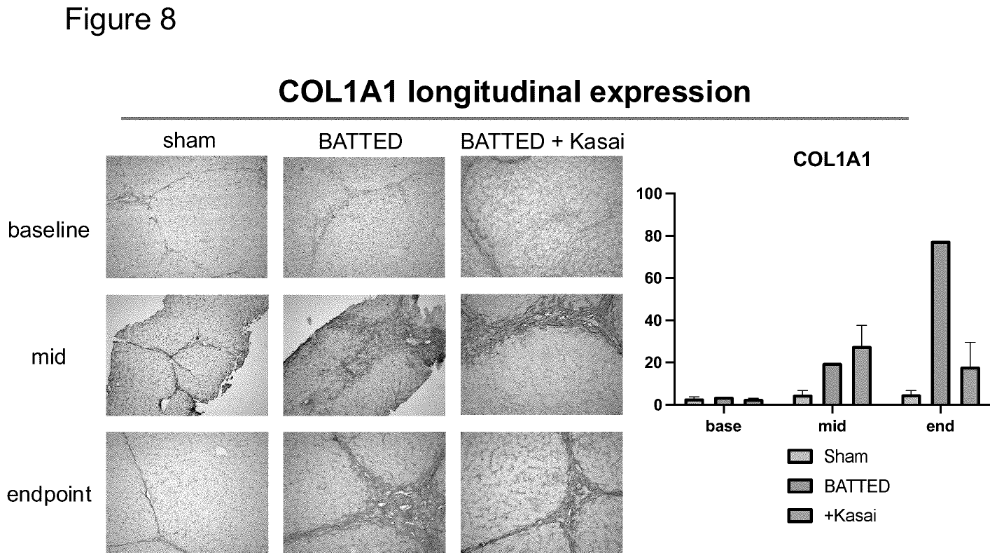

FIG. 9 shows longitudinal histological changes and αSMA expression in piglets. BATTED piglets showed severe liver fibrosis and inflammatory cell infiltration as observed in human biliary atresia. Following Kasai, piglets showed mitigation of the histological change and reduction of αSMA expression.

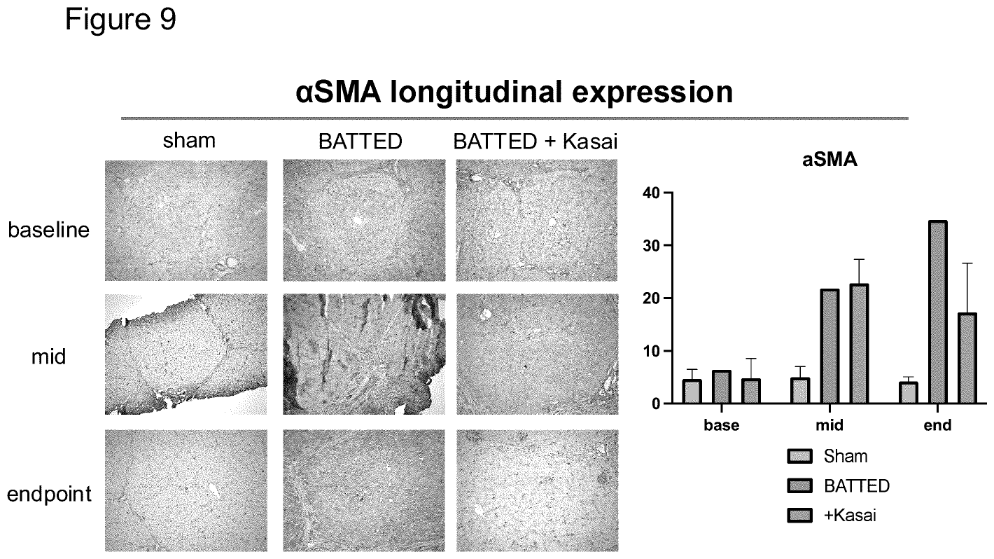

FIG. 10 shows longitudinal histological changes and CD3 expression in piglets. BATTED piglets showed severe liver fibrosis and inflammatory cell infiltration as observed in human biliary atresia. Following Kasai, piglets showed mitigation of the histological change and reduction of CD3 expression.

FIG. 11 is an illustration of the biliary atresia through targeted endothelial destruction procedure for obtaining a large animal model simulating biliary atresia of the present disclosure.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In one aspect, the present disclosure is directed to a large animal model simulating human biliary atresia. In another aspect, the present disclosure is directed to an animal model simulating human biliary atresia comprising an animal having sclerosis of intrahepatic bile ducts following injection of an agent into the intrahepatic bile ducts of the animal. Embodiments of the animal models replicate human fibrosis, cholestasis, and choleangiopathy.

The animal models simulating human biliary atresia provide reliable platforms for studying the pathophysiology of biliary atresia, enabling better understanding, drug testing, and development of therapeutic interventions. In particular, the animal models provide for complex Kasai hepatoportoenterostomy (HPE) surgery to be performed. The animal models further allow for the examination of physiological and histological changes of biliary atresia.

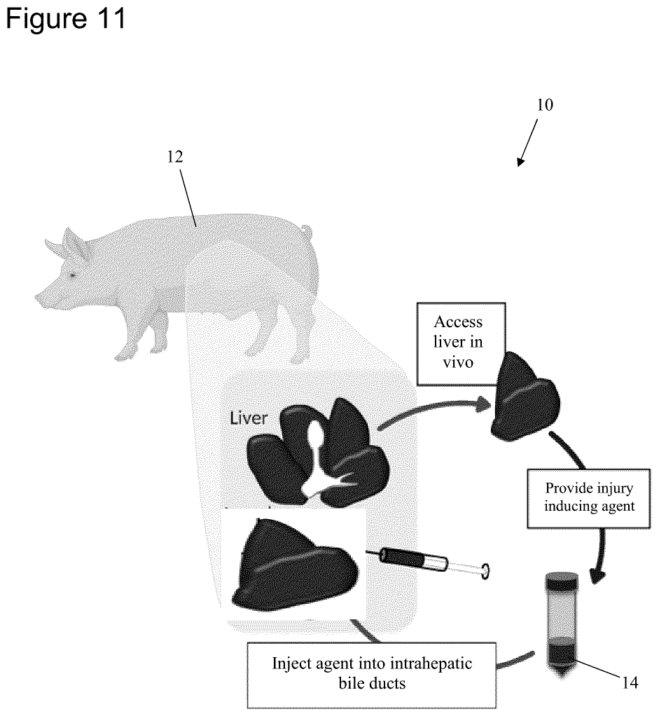

Referring to FIG. 11, an animal model for simulating human biliary atresia and methods for generating animal models are generally illustrated at 10. The animal models 10 include in vivo systems configured to recapitulate biliary atresia in humans. Thus, the animal model 10 is configured to simulate cholestasis and liver injury occurring in humans due to the condition of biliary atresia. In the illustrated embodiment, the animal model 10 includes a large animal model such that an animal 12 of the animal model includes a large animal. In one embodiment, the animal 12 is a neonatal piglet. For example, a neonatal piglet may be defined as a piglet between about 7 and about 10 days old. Additionally, the animal 12 includes pigs of other ages. Additionally, the animal 12 includes other large animals without departing from the scope of the disclosure. A “large animal” includes a species of animal whose average weight as a full grown adult is at least 46 kilograms (100 lbs.). As will be explained in greater detail below, use of a large animal in the illustrated model 10 provides a reliable platform for studying the pathophysiology of biliary atresia, enabling better understanding, drug testing, and development of therapeutic interventions for biliary atresia. Thus, the animal model 10 can be used to advance diagnostics and therapeutics for biliary atresia reducing the need for liver transplantation in subjects suffering from the condition. Alternatively, the animal model 10 includes animals other than large animals such as small animals (an animal having an average weight as a full grown adult of less than 46 kilograms) and an in vitro animal.

To simulate the condition of biliary atresia in the animal 12, the animal 12 it is treated with an agent 14 configured to cause injury to the liver of the animal 12. Thus, the agent 14 includes any substance configured to cause injury to the liver when administered to the animal 12. In one embodiment the agent 14 includes ethanol. For example, the agent 14 includes 95% ethanol. It will be understood that other agents can be administered without departing from the scope of the disclosure. Referring to FIG. 11, the agent is injected into the intrahepatic bile ducts in the liver of the animal 12. Without being bound by theory, administration of the agent 14 causes damage to the liver. In particular, administration of the agent 14 to the liver causes sclerosis of intrahepatic bile ducts. This physiological effect in the large animal 12 recapitulates the injury of bile duct proliferation in the setting of extensive hepatic fibrosis that occurs in humans. Therefore, administering the agent 14 to the large animal 12 replicates the condition of biliary atresia in the large animal.

The large animal model 10 provides a reliable platform for studying the pathophysiology of biliary atresia. Because of the size of the animal 12, more robust testing and experimentation can be performed enabling a better understanding of the condition. As a result, more effective therapeutic treatments can be devised using the animal model 10. For example, the large animal model 10 is configured such that the surgical procedure of Kasai hepatoportoenterostomy (HPE) can be performed on the animal model. Therefore, the efficacy of the surgical procedure can be studied on a model that closely replicates that of a human. This also allows for the exploration of novel diagnostics and therapeutics that improve prognosis of native livers post-HPE. Additionally, the animal model 10 allows for the examination of physiological and histological changes in the biliary atresia condition as they occur in a living animal such as a human. Thus, the condition can be studied in vivo over an extended period of time. This provides a robust system for diagnostics and therapeutic testing as well as a teaching tool for students and clinicians.

In another aspect, the present disclosure is directed a method of generating an animal model simulating human bile duct injury, the method comprising: providing an animal; and administering an agent to the animal specimen, wherein the agent causes sclerosis of intrahepatic bile ducts in the animal specimen thereby simulating human bile duct injury.

In another aspect, the present dis closure is directed to an animal model produced by the method of generating an animal model simulating human bile duct injury, the method comprising: providing an animal; and administering an agent to the animal specimen, wherein the agent causes sclerosis of intrahepatic bile ducts in the animal specimen thereby simulating human bile duct injury.

In another aspect, the present disclosure is directed a method of generating an animal model simulating human biliary atresia, the method comprising: providing an animal; and injecting an agent into intrahepatic bile ducts of the specimen, wherein the agent causes sclerosis of the intrahepatic bile ducts thereby simulating human biliary atresia.

In another aspect, the present disclosure is directed to an animal model produced by the method of generating an animal model simulating human biliary atresia, the method comprising: providing an animal; and injecting an agent into intrahepatic bile ducts of the specimen, wherein the agent causes sclerosis of the intrahepatic bile ducts thereby simulating human biliary atresia.

### EXAMPLES

The following non-limiting example is provided to further illustrate the present disclosure.

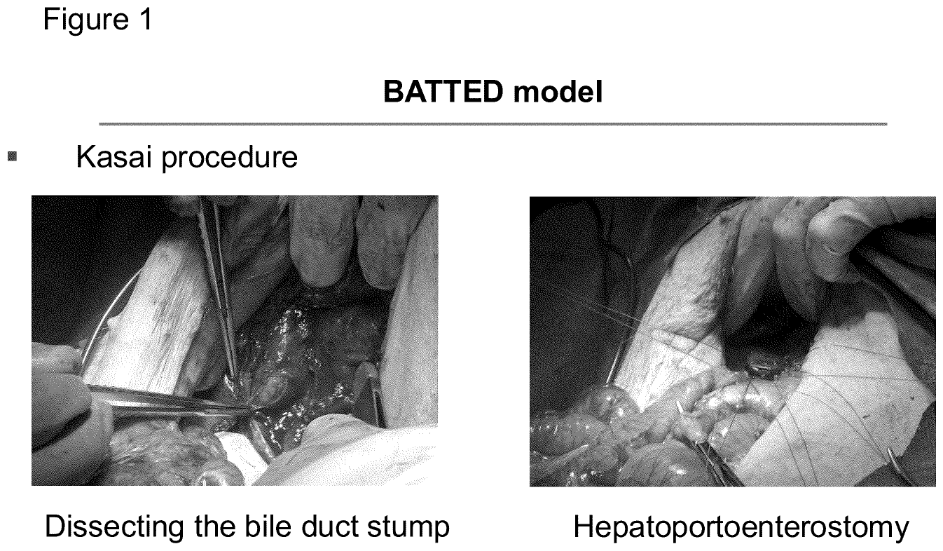

Procedure: Neonatal piglets (7 to 10 days old) were procured and underwent surgery under general anesthesia. First, a vascular access port was placed on the dorsal body, connected to the jugular vein via subcutaneous tunneling. A sub-costal incision was then made. The gall bladder was identified and after ligation of the cystic duct and common bile duct, 95% ethanol was injected into the intrahepatic bile duct to cause sclerosis of intrahepatic bile ducts, recapitulating human bile duct injury in biliary atresia. A liver biopsy was also performed. After the surgery simulating the biliary atresia condition in the piglets, weekly blood draw and bi-weekly liver biopsy were performed to assess longitudinal progression. Six weeks after surgery, a proportion of the piglets were allocated to a “Kasai group” (+Kasai) to receive hepatoportoenterostomy (HPE). For piglets in this group, a subcostal incision was made and dissection was undertaken in the hepato-portal area. The jejunum was divided at around 10 cm from the ligaments of Treitz. The distal side of the jejunum was then mobilized and the hepaticojejunostomy performed. The proximal stump was anastomosed with the jejunum about 40 cm from the hepaticojejunostomy. A drainage tube was placed under the hepaticojejunostomy. This drainage system was secured with a jacket and removed after 3 to 5 days. At around 2 months, the piglets were euthanized and tissue samples collected. Serological, histological, transcriptomic, proteomic and metabolomics analysis was performed. FIG. 1 depicts the surgical procedure.

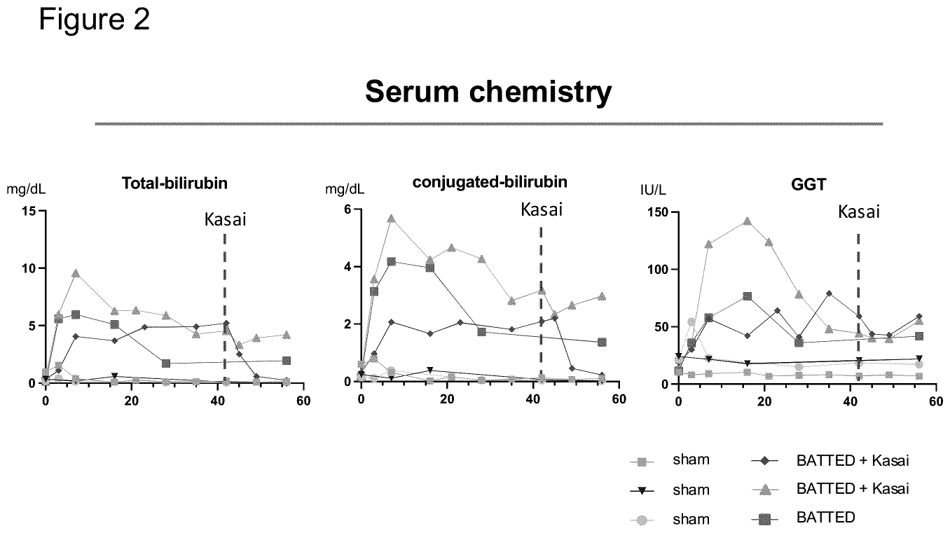

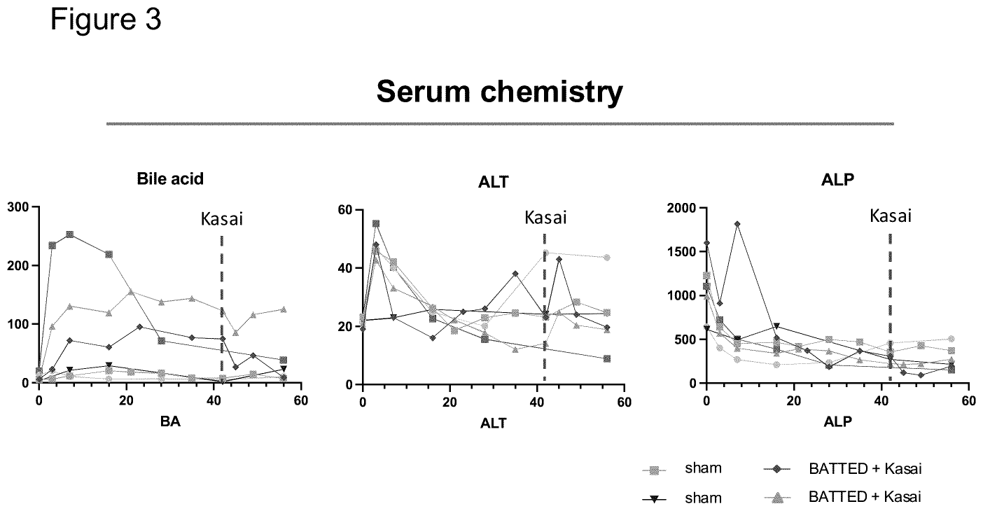

Serological analysis: Total bilirubin, conjugated bilirubin, gamma-glutamyl transferase (GGT), bile acid, alanine transaminase (ALT), and alkaline phosphatase (ALP) was examined to assess cholestasis and choleangiopathy. (See, FIGS. 2 and 3).

Transcriptomic analysis: There is no definitive gene marker for biliary atresia though some genes related to bile acid metabolism including CYP7A1, FXR, BSEP, SHP, and CAR were examined. Some potential biliary atresia markers such as GPC1, LAMC2, JAG1, COL3A1 and CXCL8 were also examined. Longitudinal expression of CK7, collagen (as detected by Sirius red staining), COL1A1, alpha-SMA (αSMA) and CD3 in BATTED piglets and BATTED+Kasai piglets was analyzed.

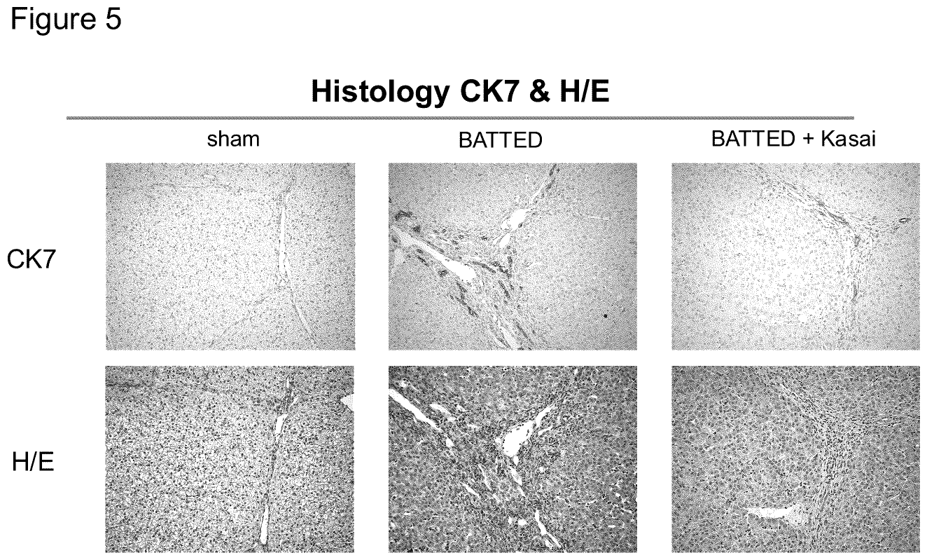

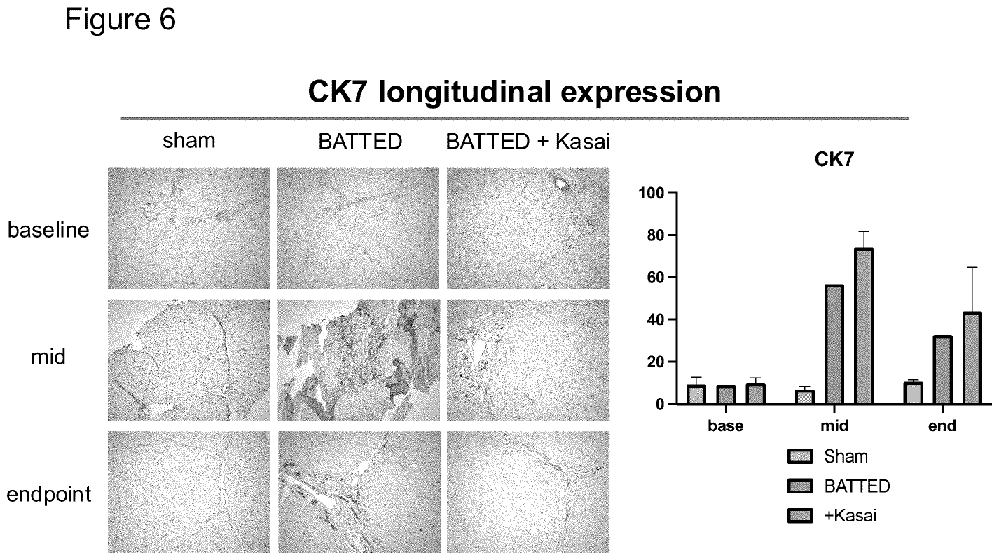

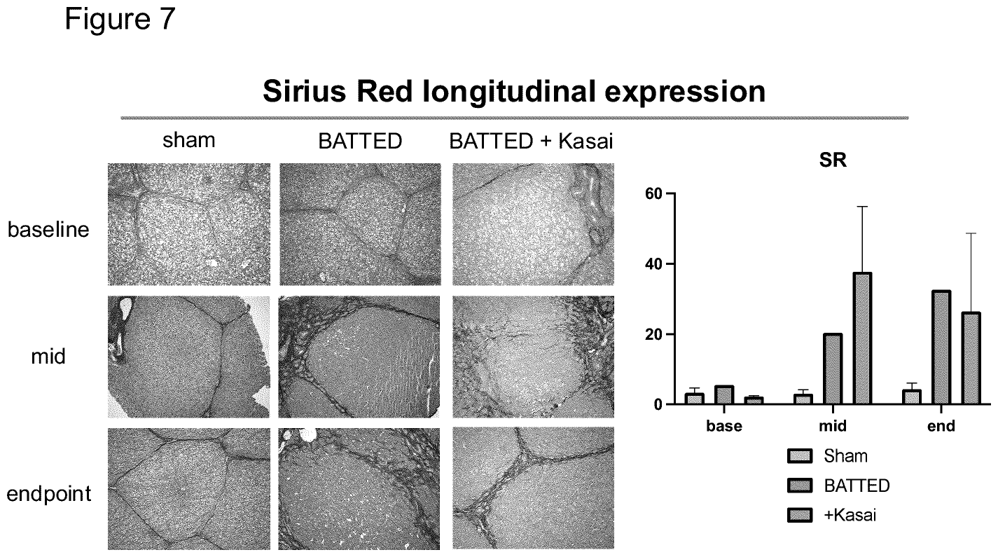

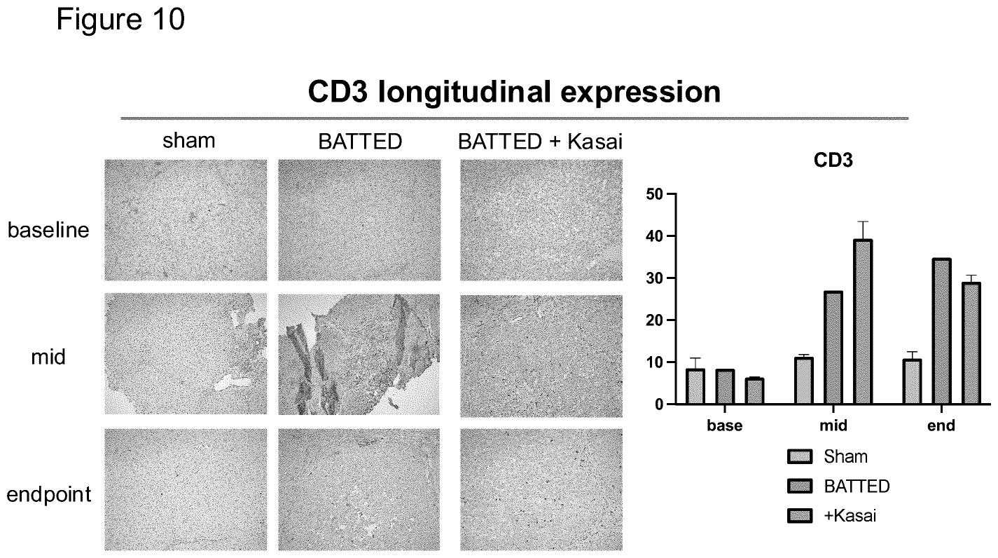

Histological analysis: H/E stain, Sirius red stain (FIG. 7) and alpha-SMA (αSMA) immunohistochemistry (IHC) were performed to assess liver fibrosis. CK7-immunohistochemistry (IHC) and H/E was performed for the degree of bile proliferation (FIGS. 5 and 6). CD3-IHC for immune cell infiltration was performed (FIG. 10).

This model well recapitulated the characteristics of cholestatic liver disease like biliary atresia in terms of serum chemistry and histology. Examination of gene expression enables studying the change of bile acid metabolism induced by BATTED procedure and Kasai surgery.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above apparatus, systems, and methods without departing from the scope of the invention, it is intended that all matter contained in the above description and shown in the accompanying drawings shall be interpreted as illustrative and not in a limiting sense.

What is claimed is:

1. A large animal model simulating human biliary atresia comprising: a large animal having sclerosis of intrahepatic bile ducts caused by administration of an agent causing bile duct injury.

2. The model of claim 1, wherein the large animal comprises a piglet.

3. The model of claim 2, wherein the large animal comprises a neonatal piglet.

4. The model of claim 1, wherein the agent comprises ethanol.

5. The model of claim 4, wherein the ethanol is injected into the intrahepatic bile ducts of the large animal.

6. A model simulating human biliary atresia comprising an animal having sclerosis of intrahepatic bile ducts following injection of an agent into the intrahepatic bile ducts of the animal.

7. The model of claim 6, wherein the agent comprises ethanol.

8. The model of claim 7, wherein the ethanol comprises 95% ethanol.

9. The model of claim 6, wherein the animal comprises a large animal.

10. The model of claim 9, wherein the large animal comprises a piglet.

11. A method of generating an animal model simulating human bile duct injury, the method comprising:
providing an animal; and
administering an agent to the animal, wherein the agent causes sclerosis of intrahepatic bile ducts in the animal thereby simulating human bile duct injury.

12. The method of claim 11, wherein the large animal comprises a piglet.

13. The method of claim 12, wherein the large animal comprises a neonatal piglet.

14. The method of claim 11, wherein the agent comprises ethanol.

15. An animal model produced by the method of claim 11.

16. A method generating an animal model simulating human biliary atresia, the method comprising:
providing an animal; and
injecting an agent into intrahepatic bile ducts of the animal, wherein the agent causes sclerosis of the intrahepatic bile ducts thereby simulating human biliary atresia.

17. The method of claim 16, wherein the agent comprises ethanol.

18. The method of claim 17, wherein the agent comprises 95% ethanol.

19. The method of claim 16, wherein the animal comprises a large animal.

20. An animal model produced by the method of claim 16.
